(19) United States
(12) Patent Application Publication
(10) Pub. No.: US 2026/0242329 A1
(43) Pub. Date: Aug. 20, 2026

(54) AN IMPROVED PROCESS FOR THE PREPARATION OF INTERMEDIATES USED IN THE PREPARATION OF DASATINIB

(71) Applicants: Harikrishna MUDDULURU, Hyderabad, Telangana (IN); Munisekhar Reddy SANNAREDDY, Hyderabad, Telangana (IN); Salivahanaiah AKKILI, Hyderabad, Telangana (IN); Mahender Reddy MARRI, Hyderabad, Telangana (IN); Ocimum Labs Private Limited, Hyderabad (IN)

(72) Inventors: Harikrishna Mudduluru, Hyderabad (IN); Munisekhar Reddy Sannareddy, Hyderabad (IN); Salivahanaiah Akkili, Hyderabad (IN); Mahender Reddy Marri, Hyderabad (IN)

(21) Appl. No.: 19/164,910

(22) PCT Filed: Mar. 12, 2024
(86) PCT No.: PCT/IB2024/052373

§ 371 Date: Sep. 12, 2025

(30) Foreign Application Priority Data
Mar. 16, 2023 (IN) 202341017625

(51) Int. Cl. C07C 209/62; C07D 417/12

(52) U.S. Cl. CPC C07C 209/62; C07D 417/12

(57) ABSTRACT

The present invention relates to an improved process for the preparation of intermediate compounds used in the preparation of Dasatinib. The present invention specifically relates to an improved process for the preparation of intermediate compound of Formula (I) used in the preparation of Dasatinib or its pharmaceutically acceptable salts.

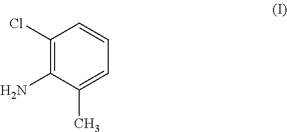

### FIELD OF THE INVENTION

The present invention relates to an improved process for the preparation of intermediate compound, 2-Chloro-6-methylaniline, used in the preparation of Dasatinib.

The present invention specifically relates to an improved process for the preparation of 2-Chloro-6-methylaniline of formula (I)

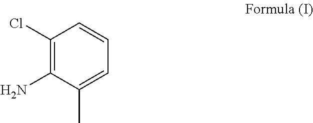

which comprises:
a) providing the sodium salt of 4-acetamido-3-methyl benzenesulphonate compound of Formula (II)

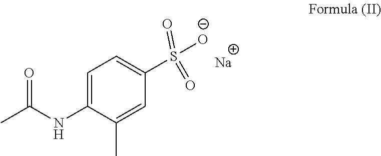

and reacting with chlorinating agent, concentrated hydrochloric acid, and Oxone® to obtain, 4-acetamido-3-chloro-5-methyl benzenesulphonate, of Formula (III),

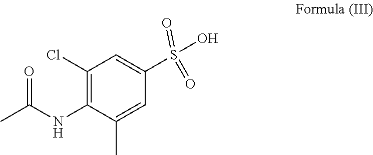 [SMILES: CC(=O)Nc1c(C)cc(S(=O)(=O)O)cc1Cl]

b) hydrolysing the acyl group of Formula (III) using concentrated hydrochloric acid to produce 4-amino-3-chloro-5-methyl benzenesulphonate of Formula (IV),

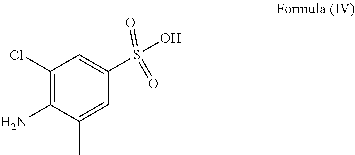

c) desulphonation of Formula (IV) using concentrated hydrochloric acid in sulfolane solvent and extraction of the product in Dichloromethane to produce the compound of Formula (I).

The present invention also specifically relates to use of intermediate compound, 2-chloro-6-methylaniline, in the preparation of Dasatinib or its pharmaceutically acceptable salts having the following structure of Formula (V).

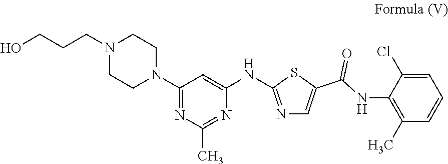 [SMILES: Cc1nc(Nc2ncc(C(=O)Nc3c(C)cccc3Cl)s2)cc(N2CCN(CCCO)CC2)n1]

### BACKGROUND OF THE INVENTION

Dasatinib is a potent, orally active, multi-targeted inhibitor of several critical oncogenic kinases. Dasatinib (DAS) was approved by the FDA in 2006 and is sold under the trade name Sprycel. (Cryst. Growth Des. 2012, 12, 2122-2126) Chemically Dasatinib is known as [N-(2-chloro-6-methylphenyl)-2-[[6-[4-(2-hydroxyethyl)-1-piperazinyl]-2-methyl-4-pyrimidinyl]amino]-5-thiazole carboxamide monohydrate], and has the following chemical structure.

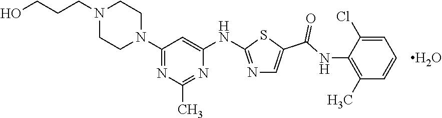

U.S. Pat. No. 6,596,746 B1 discloses the compound, N-(2-chloro-6-methylphenyl)-2-[[6-[4-(2-hydroxyethyl)-1-piperazinyl]-2-methyl-4-pyrimidinyl]amino]-5-thiazole carboxamide and salts thereof, and methods of using such compounds in the treatment of protein tyrosine kinase-associated disorders such as immunologic and oncologic disorders. This patent also discloses the preparation process of the above compound using 2-Chloro-6-methylaniline as raw material.

U.S. Pat. No. 7,491,725 B2 discloses the monohydrate form of the N-(2-chloro-6-methylphenyl)-2-[[6-[4-(2-hydroxyethyl)-1-piperazinyl]-2-methyl-4-pyrimidinyl]amino]-5-thiazolecarboxamide, which is known as Dasatinib.

As the compound, 2-Chloro-6-methylaniline, is an important intermediate to prepare the Dasatinib, various synthetic procedures have been developed that suitable for industrial production, ecofriendly, cost-effective and high purity API. The basic and complicated step in the preparation process is Regio-selective chlorination.

GB 190901245 A discloses the preparation of the intermediate compound, 3-chloro-2-amino-1-methyl-benzene-5-sulphonic acid by chlorinating 2-acidy-amino-1-methyl-benzene-5-sulphonic acid with chlorine in aqueous solution and splitting off the acedyl group. The process disclosed is given below:

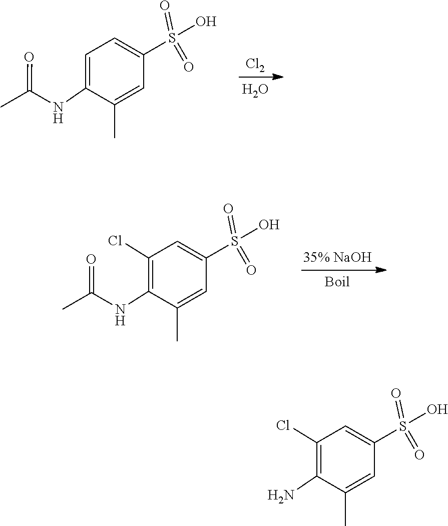

The disadvantages associated with the above process are usage of poisonous Chlorine gas, produces lot of effluents and high operation time (6-8 hours) in industry.

J. Am. Chem. Soc. 1946, 68, 861-864 discloses the process for analogous compound, 3-nitro-2-amino-toluene, where the amine moiety is replaced by chlorine via diazotonium salt intermediate compound with sodium nitrite and cuprous chloride. Similar process is used in later stages to prepare 2-amino-3-chlorotoluene. The process disclosed is given below.

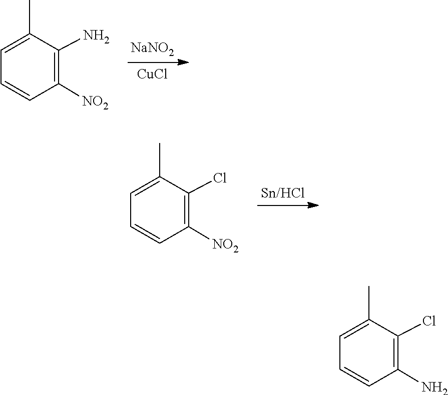

The disadvantages associated with the above process are tedious process, high impurities and multiple steps.

J. Am. Chem. Soc. 1955, 77, 21, 5700-5702 discloses the process, where the chlorination process is carried out using Sodium chlorate and conc.HCl. Sodium chlorate acts as oxidizing agent and reduces HCl into Chlorine gas. The elimination of sulphonic acid is done by using sulfuric acid at higher temperatures. The process disclosed for the preparation of 2-amino-3-chlorotoluene is given below.

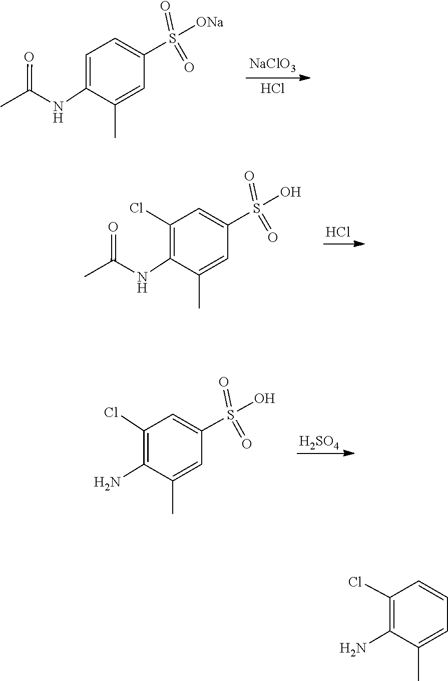

The disadvantages associated with the above process are sodium chlorite is strong oxidizing agent, highly exothermic reaction, release lot of effluents and also formation of chlorine dioxide gas during the process, (https://doi.org/10.4028/www.scientific.net/AMR.634-638.546) further, excess oxidizing agent should be neutralized to continue the reaction.

CN 102079688 B discloses the process, which involves the chlorination after condensing with urea and blocking para-position with sulfonic acid. However, the process has more stages and yield is low, i.e., 39%. The process disclosed for the preparation of 2-amino-3-chlorotoluene is given below.

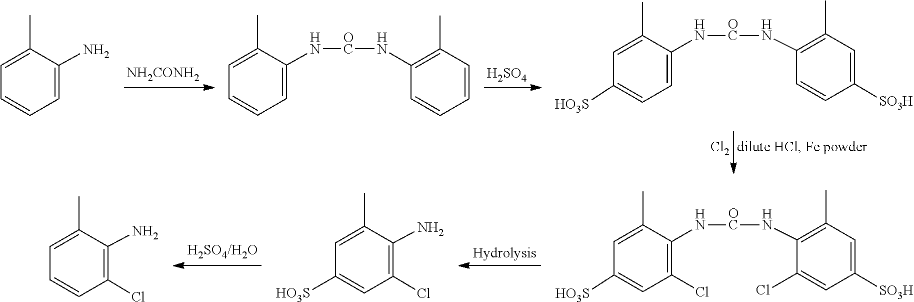

CN 110015963 A discloses the process using the chlorinating agent, HCl, in presence of oxidizing agent, (hydrogen peroxide or Zinc Chloride), and followed by removal of sulphonic moiety. The process disclosed for the preparation of 2-amino-3-chlorotoluene is given below.

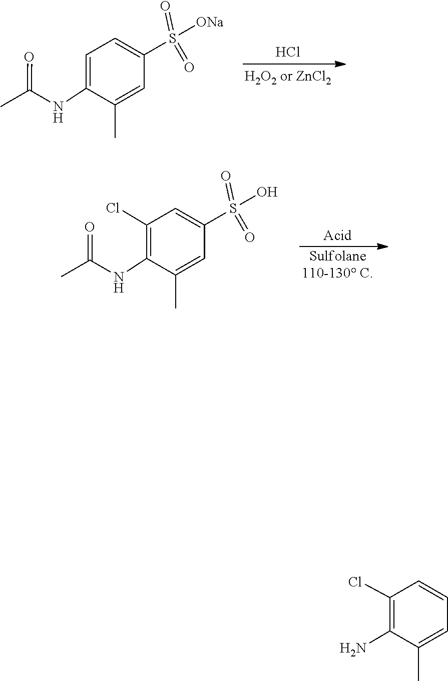

The disadvantages associated with the above process are hydrogen peroxide, being strong oxidizing agent, release lot of effluents and also exothermic reaction, further, excess oxidizing agent should be quenched with other reagents neutralising to continue the reaction, which can influence the purity and the yield. The yield of the above process is low, i.e. 60%.

In order to improve the yield, Regio-selective chlorination, product purity, usage of environmental friendly reagents, nitrite impurities free, minimize the process steps such as to eliminate quenching strong oxidizing agents upon the completion of chlorination reaction, to avoid exothermic reaction, and to reduce gas effluents during reaction, different methods have been tried for these three steps of chlorination, de-carboxylation and removal of sulphonic acid moiety to come-up with improved process for the preparation of Dasatinib intermediate, 2-chloro-6-methylaniline.

The present inventors have surprisingly found new improved process for Dasatinib intermediate synthesis, that involve Chlorination process using Oxone® and further, the extraction of 2-chloro-6-methylaniline in order to achieve high yield and pure Dasatinib intermediate compound.

Oxone® (KHSO₅_{^{−}}1/2 KHSO₄_{^{−}}1/2 K₂SO₄) has been widely used as a green, cheap, and safe oxidant because it generates K₂SO₄ as the only oxidant-derived byproduct. Oxone® has not only admirable bench-stability for storage and transportation but also exceptional reactivity towards halide (e.g., bromide and chloride) oxidation under weakly acidic/basic or even neutral conditions which is advantageous over related halide oxidation with hydrogen peroxide or oxygen under either strong acid (HCl or HBr) or transition metal catalysis. (Nat Commun 10, 4754 (2019)—doi.org/10.1038/s41467-019-12768-4) Sulfolane is a dipolar aprotic industrial solvent, very stable at elevated temperatures and easily recoverable.

The process of the present invention not only avoids the use of highly toxic reagents, unstable and/or expensive reagents, lengthy process by avoiding steps to quench oxidizing agents but also leads to the desire product, in shorter reaction times and with higher productivity (75% yield) and 99.8% purity than prior art processes. These features make the process of the invention cost-efficient making the process highly suitable for industrial scale up production.

### OBJECTIVE OF THE INVENTION

The main objective of the present invention is to provide an improved process for the preparation of intermediate compounds used in the preparation of Dasatinib.

Another objective of the present invention is to provide an improved process for the preparation of intermediate compounds of formula (I) with 99.8% purity, which is used in the preparation of Dasatinib or its pharmaceutically acceptable salts.

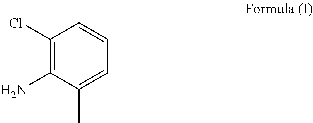

Still another preferred objective of the present invention is to provide use of intermediate compound of Formula (I) in the preparation of Dasatinib or its pharmaceutically acceptable salts having the following structure of Formula (V).

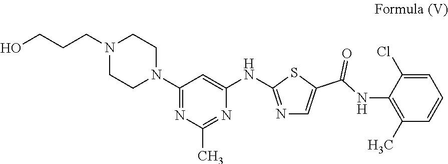 [SMILES: Cc1nc(Nc2ncc(C(=O)Nc3c(C)cccc3Cl)s2)cc(N2CCN(CCCO)CC2)n1]

### SUMMARY OF THE INVENTION

Accordingly, the present invention provides an improved process for the preparation of intermediate compound of formula (I)

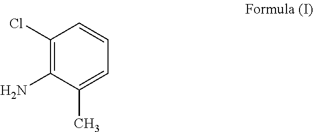

which comprises:
a) providing the sodium salt of 4-acetamido-3-methyl benzenesulphonate compound of Formula (II)

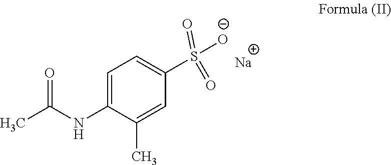

and reacting with chlorinating agent, and oxone in the presence or absence of a polar solvent to obtain an intermediate compound of Formula (III),

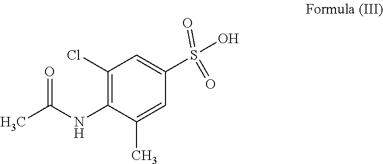 [SMILES: CC(=O)Nc1c(C)cc(S(=O)(=O)O)cc1Cl]

b) hydrolysing the acyl group of intermediate compound of Formula (I) using an acid to produce the intermediate compound of Formula (IV),

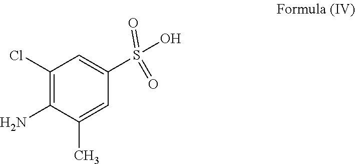

c) desulphonation of intermediate compound of Formula (IV) using an acid in solvent and extraction of the product using a solvent to produce the intermediate compound of Formula (I).

In another embodiment the present invention provides an improved process for the preparation of intermediate compound of Formula (I)

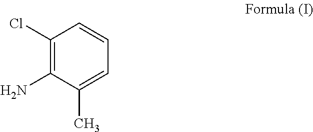

which comprises:
a) providing the sodium salt of 4-acetamido-3-methyl benzenesulphonate compound of Formula (II)

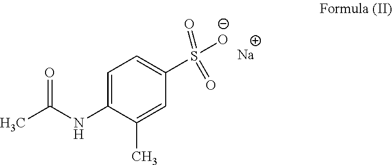

and reacting with chlorinating agent, concentrated hydrochloric acid and Oxone® to obtain an intermediate compound of Formula (III),

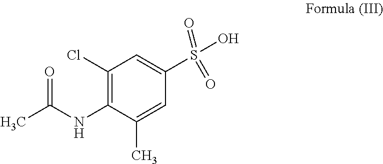 [SMILES: CC(=O)Nc1c(C)cc(S(=O)(=O)O)cc1Cl]

b) hydrolysing the acyl group of intermediate compound of Formula (I) using concentrated hydrochloric acid to produce the intermediate compound of Formula (IV),

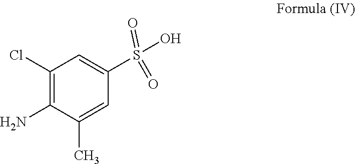

c) desulphonation of intermediate compound of Formula (IV) using concentrated hydrochloric acid in sulfolane solvent and extraction of the product in Dichloromethane to produce the intermediate compound of Formula (I),

In another embodiment, the present invention also provides an improved process for the preparation of Dasatinib or its pharmaceutically acceptable salts of Formula (V)

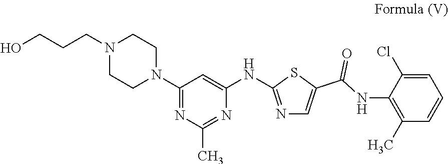 [SMILES: Cc1nc(Nc2ncc(C(=O)Nc3c(C)cccc3Cl)s2)cc(N2CCN(CCCO)CC2)n1]

using compound of formula (I) prepared by a process of the present invention.

### DETAILED DESCRIPTION OF THE INVENTION

The term “comprising”, which is synonymous with “including”, “containing”, or “characterized by” here is defined as being inclusive or open-ended, and does not exclude additional, unrecited elements or method steps, unless the context clearly requires otherwise.

In a specific embodiment, the chlorination reaction of compound of Formula (II) with chlorinating agent and polar solvent in presence of an oxidizing agent, Oxone®, is carried out at 50° C. and stir for 2 hours. After completion of the reaction, the mixture was cooled to 30° C. and filtered the solids of the compound of Formula (III). The reaction may be carried out at a temperature in the range of 40° C. to 80° C. for a duration of 1 to 4 hours. Further, Chlorinating agents can be used in the process are salts like NaCl, KCl and NH₄Cl in polar solvent. Organic polar solvent as used herein is selected from alcohol solvents, chlorinated solvents, acetic acid, ethanol, isopropanol, dichloroethane, toluene, cyclohexane or its mixture. However, use of HCl as chlorinating agent yields 86% chlorinated compound of Formula (III) with 99.5% purity.

Hydrolysis of acyl group of the compound of formula (III) is carried out using conc. HCl at 110° C. and stirred for 2 hours. After completion of the reaction the compound of Formula (IV) washed with IPA and dried. The reaction may be carried out at a temperature in the range of 90° C. to 120° C. 1 to 5 hours. Further, acid can be used such as HCl, H₂SO₄ and H₃PO₄. However, Conc. HCl yields high productivity with 86% yield and 99.7% purity of acylated compound of Formula (IV).

Removal of para-position blocking moiety, sulphonic acid, is carried out by desulphonation using acid in presence of sulfolane solvent at 120° C. for 24 hours. After completion of reaction, mass neutralized with base and extracted the compound of Formula (I) using Dichloromethane. The desulphonation may be carried out using acids such as HCl, H₂SO₄ and H₃PO₄ or water at higher temperatures. However, desulphonation using HCl at 120° C. for 24 hours and extraction of the compound formula (I) in Dichloromethane produced good yield (75%) and high purity (99.8%) compound of Formula (III), 2-chloro-6-methylaniline.

In yet another embodiment, the addition of HCl and Oxone® mixture to 4-acetamido-3-methyl benzenesulphonate used in the present invention is done in the range of 1-4 hours.

The solvent as used herein are selected from water or “alcohol solvents” such as methanol, ethanol, n-propanol, isopropanol, n-butanol and t-butanol and the like or “hydrocarbon solvents” such as benzene, toluene, xylene, heptane, hexane and cyclohexane and the like or “ketone solvents” such as acetone, ethyl methyl ketone, diethyl ketone, methyl tert-butyl ketone, isopropyl ketone and the like or “esters solvents” such as methyl acetate, ethyl acetate, propyl acetate, isopropyl acetate, n-butyl acetate, isobutyl acetate, sec-butyl acetate, and the like or “nitrile solvents” such as acetonitrile, propionitrile, butyronitrile and isobutyronitrile and the like or “ether solvents” such as di-tert-butylether, dimethylether, diethylether, diisopropyl ether, 1,4-dioxane, methyltert-butylether, ethyl tert-butyl ether, tetrahydrofuran, 2-methyl tetrahydrofuran, 2-methoxyethanol and dimethoxyethane, or “Amide solvents” such as formamide, DMF, DMAC, N-methyl-2-pyrrolidone, N-methylformamide, 2-pyrrolidone, 1-ethenyl-2-pyrrolidone, haloalkanes such as dichloromethane, 1,2-dichloroethane and chloroform, “amine solvents” selected from diethylenetriamine, ethylenediamine, morpholine, piperidine, pyridine, quinoline, tributylamine, diisopropyl amine, sulfolane, water and/or mixtures thereof.

The term “salts” as used herein refers to salts which are known to be non-toxic and are commonly used in the pharmaceutical literature. Typical inorganic acids used to form such salts include hydrochloric, hydrobromic, hydroiodic, nitric, sulfuric, phosphoric, hypophosphoric, and the like. Salts derived from organic acids, such as aliphatic mono and dicarboxylic acids, phenylsubstituted alkanoic acids, hydroxyalkanoic and hydroxyalkandioic acids, aromatic acids, aliphatic and aromatic sulfonic acids, may also be used. Such salts thus include acetate, phenylacetate, trifluoroacetate, acrylate, ascorbate, benzoate, chlorobenzoate, dinitrobenzoate, hydroxybenzoate, methoxybenzoate, methylbenzoate, o-acetoxybenzoate, naphthalene-2-benzoate, bromide, isobutyrate, phenylbutyrate, beta-hydroxybutyrate, chloride, cinnamate, citrate, formate, fumarate, glycolate, heptanoate, lactate, maleate, hydroxymaleate, malonate, mesylate, nitrate, oxalate, phthalate, phosphate, monohydro genphosphate, dihydrogenphosphate, metaphosphate, pyrophosphate, propionate, phenylpropionate, salicylate, succinate, sulfate, bisulfate, pyrosulfate, sulfite, bisulfite, sulfonate, benzenesulfonate, p-bromophenylsulfonate, chlorobenzenesulfonate, ethanesulfonate, 2-hydroxyethanesulfonate, methanesulfonate, naphthalene-1-sulfonate, naphthalene-2-sulfonate, p-toluenesulfonate, xylenesulfonate, tartarate, and the like.

In another preferred embodiment, a process for the preparation of compound of Formula (I) which yields the compounds with high chemical purity.

In yet another preferred embodiment, the present invention provides use of intermediate compounds of Formula (I) in the preparation of Dasatinib or its salts. The intermediates formed in the present invention may or may not be isolated. Any of the above reactions may be carried out in-situ reactions to obtain intermediate compound of Formula (I).

The present invention is further illustrated by the following examples which are provided merely to be exemplary of the inventions and is not intended to limit the scope of the invention. Certain modifications and equivalents will be apparent to those skilled in the art and are intended to be included within the scope of the present invention.

### EXAMPLES

### Example 1: Preparation of 4-acetamido-3-chloro-5-methylbenzenesulphonic acid (III)

Method A: The sodium 4-acetamido-3-methylbenzenesulfonate (II), 100 g was added into 300 ml. conc. HCl at 30° C. after that the Oxone® (130 g) was added portion wise. While adding the Oxone® temperature of the solution was not exceeded to 45° C. After addition of Oxone® the temperature rose to 50° C. and stirred for two hours. The mixture was cooled to 30° C. and filtered the solids. The crystals are dried at 100° C. for 6 h gives 86% yield and 99.5% purity of chlorinated compound.

Method B: The acetylated salt, 100 g was added into 300 mL of acetonitrile and 26 g of NaCl at 30° C. after that the Oxone® (130 g) was added portion wise. While adding the Oxone® temperature of the solution was not exceeded to 45° C. After addition of Oxone® the temperature rose to 50° C. and stirred for two hours. The solvent was distilled out under reduced pressure at 50° C. then 300 mL of water was added and cooled to 30° C. and filtered the solids. The crystals are dried at 100° C. for 6 h gives 36% yield and 60% purity of chlorinated compound.

Method C: The acetylated salt, 100 g was added into 300 mL of acetonitrile and 23.4 g of NH₄Cl at 30° C. after that the Oxone® (130 g) was added portion wise. While adding the Oxone® temperature of the solution was not exceeded to 45° C. After addition of Oxone® the temperature rose to 50° C. and stirred for two hours. The solvent was distilled out under reduced pressure at 50° C. then 300 mL of water was added and cooled to 30° C. and filtered the solids. The crystals are dried at 100° C. for 6 h gives 51% yield and 71% purity of chlorinated compound.

### Example-2: Preparation of 4-amino-3-chloro-5-methylbenzenesulphonic acid (IV)

Method A: The chlorinated compound, 100 g was added into 500 ml. conc. HCl at 30° C. after that the temperature rose to 110° C. and stirred for two hours. The mixture was cooled to 30° C. and filtered the solids. The wet solid was taken into 300 mL of isopropyl alcohol and stir for 2 h at 30° C. and filtered the solids. The crystals dried at 60° C. for 6 h gives 86% yield and 99.7% purity of deacylated compound.

Method B: The chlorinated compound, 100 g was added into 500 mL of 50% H₂SO₄ at 30° C. after that the temperature rose to 110° C. and stirred for two hours. The mixture was cooled to 30° C. and filtered the solids. The wet solid was taken into 300 mL of isopropyl alcohol and stir for 2 h at 30° C. and filtered the solids. The crystals dried at 60° C. for 6 h gives 75% yield and 99.3% purity of deacylated compound.

Method C: The chlorinated compound, 100 g was added into 500 mL of 50% H₃PO₄ at 30° C. after that the temperature rose to 110° C. and stirred for two hours. The mixture was cooled to 30° C. and filtered the solids. The wet solid was taken into 300 mL of isopropyl alcohol and stir for 2 h at 30° C. and filtered the solids. The crystals dried at 60° C. for 6 h gives 70% yield and 98.1% purity of deacylated compound.

### Example-3: Preparation of 2-chloro-6-methylaniline (I)

Method A: The deacylated compound, 100 g was dissolved in 310 mL of sulfolane and 80 mL conc. HCl and raised the reaction mass to 120° C. and stir for 24 h. The reaction mass neutralize with NaOH solution and extract the compound with Dichloromethane (among other solvents) provided good yield and better quality of product) gives 75% yield and 99.8% purity of 2-Chloro-6-methylaniline.

Method B: The deacylated compound, 100 g was dissolved in 310 mL of sulfolane and 80 mL of 50% H₂SO₄ and raised the reaction mass to 120° C. and stir for 24 h. The reaction mass neutralize with NaOH solution and extract the compound with Dichloromethane gives 60% yield and 99.6% purity of 2-Chloro-6-methylaniline.

Method C: The deacylated compound, 100 g was dissolved in 310 mL of sulfolane and 80 mL of water and raised the reaction mass to 120° C. and stir for 24 h. The reaction mass neutralize with NaOH solution and extract the compound with Dichloromethane gives 45% yield and 99.4% purity of 2-Chloro-6-methylaniline.

1: An improved process for the preparation of intermediate compound of Formula (I) 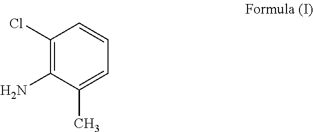
which comprises:
a) providing the sodium salt of 4-acetamido-3-methyl benzenesulphonate compound of Formula (II) 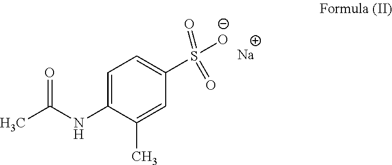
and reacting with chlorinating agent, and oxone in the presence or absence of a polar solvent to obtain an intermediate compound of Formula (III), 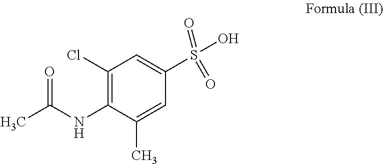 [SMILES: CC(=O)Nc1c(C)cc(S(=O)(=O)O)cc1Cl]
b) hydrolysing the acyl group of intermediate compound of Formula (I) using an acid to produce the intermediate compound of Formula (IV), 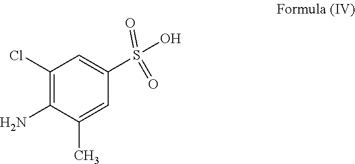
c) desulphonation of intermediate compound of Formula (IV) using an acid in solvent and extraction of the product using a solvent to produce the intermediate compound of Formula (I).

2: An improved process for the preparation of intermediate compound of Formula (I) 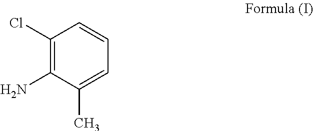
which comprises:
a) providing the sodium salt of 4-acetamido-3-methyl benzenesulphonate compound of Formula (II) 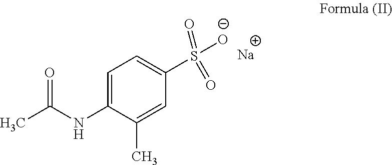
and reacting with chlorinating agent, concentrated hydrochloric acid and Oxone® to obtain an intermediate compound of Formula (III), 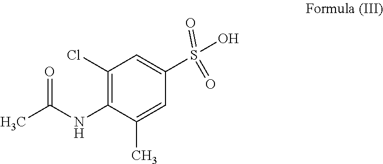 [SMILES: CC(=O)Nc1c(C)cc(S(=O)(=O)O)cc1Cl]
b) hydrolysing the acyl group of intermediate compound of Formula (I) using concentrated hydrochloric acid to produce the intermediate compound of Formula (IV), 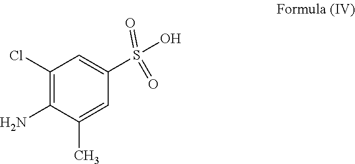
c) desulphonation of intermediate compound of Formula (IV) using concentrated hydrochloric acid in sulfolane solvent and extraction of the product in dichloromethane to produce the intermediate compound of Formula (I).

3: The process as claimed in claim 1, wherein the intermediate compound of formula (I) is utilised in the preparation of Dasatinib or its pharmaceutically acceptable salts of Formula (V)

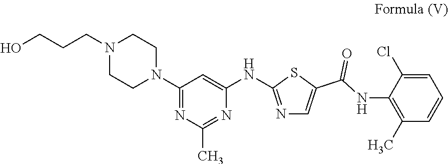 [SMILES: Cc1nc(Nc2ncc(C(=O)Nc3c(C)cccc3Cl)s2)cc(N2CCN(CCCO)CC2)n1]

4: The process as claimed in claim 1, wherein the chlorinating agent is HCl, NaCl, KCl, NH₄Cl.

5: The process as claimed in claim 1, wherein the solvent is methanol, ethanol, n-propanol, isopropanol, n-butano, t-butanol, benzene, toluene, xylene, heptane, hexane, acetone, ethyl methyl ketone, diethyl ketone, methyl tert-butyl ketone, isopropyl ketone, methyl acetate, ethyl acetate, propyl acetate, isopropyl acetate, n-butyl acetate, isobutyl acetate, sec-butyl acetate, acetonitrile, propionitrile, butyronitrile and isobutyronitrile, di-tert-butylether, dimethylether, diethylether, diisopropyl ether, 1,4-dioxane, methyltert-butylether, ethyl tert-butyl ether, tetrahydrofuran, 2-methyl tetrahydrofuran, 2-methoxyethanol, dimethoxyethane, formamide, DMF, DMAC, N-methyl-2-pyrrolidone, N-methylformamide, 2-pyrrolidone, 1-ethenyl-2-pyrrolidone, dichloromethane, 1,2-dichloroethane, chloroform, diethylenetriamine, ethylenediamine, morpholine, piperidine, pyridine, quinoline, tributylamine, diisopropyl amine, sulfolane and water.

6: The solvent as claimed in claim 5, wherein the solvent is methanol, ethanol, n-propanol, isopropanol, n-butano, t-butanol, acetonitrile, propionitrile, butyronitrile, isobutyronitrile, dichloromethane, 1,2-dichloroethane, chloroform, sulfolane and water.

7: The process as claimed in claim 1, wherein the hydrolysis is carried out using conc. HCl, H₂SO₄ and H₃PO₄.

8: The process as claimed in claim 1, wherein the desulphonation is carried out using acid selected from HCl, H₂SO₄ and H₃PO₄.

9: The process as claimed in claim 2, wherein the intermediate compound of formula (I) is utilised in the preparation of Dasatinib
or its pharmaceutically acceptable salts of Formula (V) 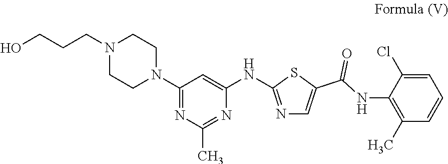 [SMILES: Cc1nc(Nc2ncc(C(=O)Nc3c(C)cccc3Cl)s2)cc(N2CCN(CCCO)CC2)n1]
